(19) United States
(12) Patent Application Publication
(10) Pub. No.: US 2016/0151078 A1
(43) Pub. Date: Jun. 2, 2016

(54) CATHETER

(71) Applicant: ASAHI INTECC CO., LTD., Nagoya-shi (JP)

(72) Inventor: Yuya KANAZAWA, Seto-shi (JP)

(21) Appl. No.: 14/934,683

(22) Filed: Nov. 6, 2015

(30) Foreign Application Priority Data
Nov. 27, 2014 (JP) 2014-239529

(51) Int. Cl. A61B 17/22; A61M 25/00

(52) U.S. Cl. CPC A61B 17/22; A61M 25/001; A61M 25/0043; A61B 2017/22094; A61B 2017/00526

(57) ABSTRACT

A catheter including a hollow cylindrical main body comprising at least one metallic wire, and a hollow distal end tip that is integrally formed with a distal end of the cylindrical main body. The distal end tip remains securely fixed to the cylindrical main body even under application of a large torque force.

### CROSS REFERENCE TO RELATED APPLICATION

This application claims priority to Japanese Application No. 2014-239529 filed on Nov. 27, 2014, the content of which is incorporated by reference herein in its entirety.

### BACKGROUND

The disclosed embodiments relate to a medical device. Specifically, the disclosed embodiments relate to a catheter suitable for passing through a hard lesion or the like.

Conventionally, a catheter is used for treating the inner lumen of a blood vessel or the like. For example, when a lumen of a blood vessel or the like includes an occluded portion, a stenosed portion, or the like, a catheter can be passed through the occluded portion or the stenosed portion to restore the lumen. The catheter must be flexible in order to progress through the lumen to a target region. In addition, the distal end of the catheter must be able to easily pass into the occluded portion, the stenosed portion, or the like (that is, the catheter must have excellent “passing performance”), and must have enough rigidity to pass through the inside of the occluded portion, the stenosed portion, or the like.

A known intraosseous catheter (Japanese Patent Application Laid-Open No. 2007-244492) has a flexible main body and a cylindrical metallic distal end tip that lends rigidity to a distal end of the catheter. In use, the intraosseous catheter is inserted into a medullary cavity of a bone.

However, when a catheter is inserted into a hard lesion such as an occluded portion, a stenosed portion, or the like, the catheter is rotated or moved forward or backward in a state where the distal end of the catheter is in contact with the occluded portion or the stenosed portion and is hard to move. Thus, a torque force is applied to the distal end of the catheter. In the case of the conventional catheter with a distal end tip attached to the main body as a separate member, when the torque force is large, the distal end tip easily separates from the main body.

### SUMMARY

An object of the disclosed embodiments is to provide a catheter that is excellent in flexibility and in passing performance into an occluded portion, a stenosed portion, or the like, and whose distal end tip remains securely fixed to the main body even under application of a large torque force.

A catheter of the disclosed embodiments includes a hollow cylindrical main body that is formed of at least one metallic wire, and a hollow distal end tip that is formed at a distal end of the cylindrical main body by melting a distal portion of the at least one metallic wire of the cylindrical main body so as to fix together adjacent windings of the at least one metallic wire in the distal portion. Because the distal end tip is formed by melting the distal portion of the at least one metallic wire of the cylindrical main body, the distal end tip and the cylindrical main body are integrally formed. This prevents the distal end tip from separating from the cylindrical main body when exposed to high torque forces. Moreover, the flexibility of the distal end of the catheter is not deteriorated.

The cylindrical main body may be formed by winding a metallic wire into a helical coil structure around a longitudinal axis of the catheter. When the cylindrical main body is formed in this manner, the cylindrical main body has excellent flexibility. As a result, there is an increased difference in flexibility between the distal end tip and the cylindrical main body. In the conventional catheter having the metallic tip and the cylindrical main body as separate members, this would increase the risk of the distal end tip separating from the cylindrical main body under application of torque forces. In the catheter of the disclosed embodiments, however, the distal end tip and the cylindrical main body are integrally formed, thereby preventing the distal end tip from separating from the cylindrical main body.

The cylindrical main body may be formed by winding a plurality of metallic wires into a helical coil structure around the longitudinal axis of the catheter, thereby further improving the flexibility of the cylindrical main body. Here too, the distal end tip and the cylindrical main body are integrally formed, thereby preventing the distal end tip from separating from the cylindrical main body under application of torque forces. In addition, the number of ends of the metallic wires that are positioned at the distal end of the cylindrical main body is plural, and the number of the metallic wires joined by melting during formation of the distal end tip is increased. Therefore, the distal end tip is formed easily and more securely.

### BRIEF DESCRIPTION OF THE DRAWINGS

FIG. 1 is a side view illustrating a catheter of the disclosed embodiments;

FIG. 2 is a cross-sectional view illustrating a distal end of the catheter of FIG. 1;

FIG. 3 is a cross-sectional view illustrating a distal end of a catheter of the disclosed embodiments;

FIG. 4 is a side view illustrating a catheter of the disclosed embodiments;

FIG. 5 is a cross-sectional view illustrating a distal end of the catheter of FIG. 4; and

FIG. 6 is a cross-sectional view illustrating a distal end of a catheter of the disclosed embodiments.

### DETAILED DESCRIPTION OF EMBODIMENTS

Catheters of the disclosed embodiments will be described with reference to the drawings. However, the invention is not limited thereto. In the drawings, the left side corresponds to a distal end of the catheter to be inserted into a body, while the right side corresponds to a proximal end of the catheter to be operated by a technician such as a physician. The proximal end remains exposed outside the body during operation without being inserted into the body. Note that the sizes of components in the drawings are exaggerated to facilitate understanding.

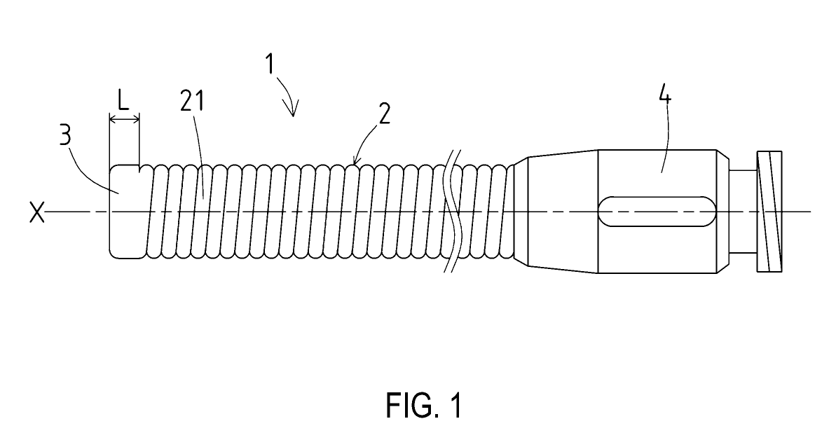

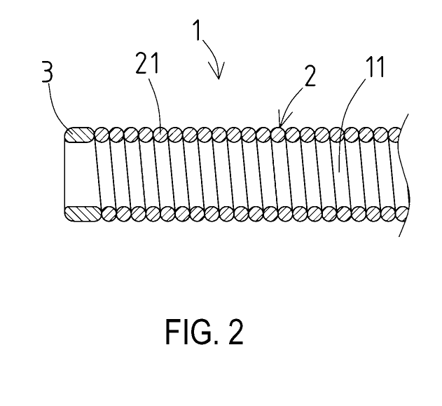

FIG. 1 and FIG. 2 are diagrams illustrating a catheter (catheter 1) of the disclosed embodiments. FIG. 1 is a side view of the catheter 1, and FIG. 2 is a cross-sectional view illustrating a distal end of the catheter 1.

The catheter 1 includes a hollow cylindrical main body 2, and a hollow distal end tip 3 formed at a distal end of the cylindrical main body 2. A connector 4 is shown connected to a proximal end of the cylindrical main body 2. The connector 4 is not inserted into the body during operation.

In use, the catheter 1 is inserted into a body lumen of a blood vessel or the like. The catheter 1 may be 600 mm to 1500 mm in length and 0.5 mm to 1.5 mm in diameter, and includes a lumen 11 passing through the catheter 1 from a proximal end to the distal end thereof. A guide wire, another catheter, or the like, which are used together with the catheter 1 in treatment, may be inserted into the lumen 11.

The cylindrical main body 2 is a hollow long body extending in a longitudinal direction of the catheter 1. In FIG. 1, the cylindrical main body 2 extends along substantially the whole length of the portion of the catheter 1 to be inserted into the body. However, it is sufficient that the cylindrical main body 2 constitutes at least a distal end portion of the catheter 1. Thus, the cylindrical main body 2 may constitute the distal end portion of the catheter 1, and another cylindrical body may be connected to the proximal end of the cylindrical main body 2, for example.

The cylindrical main body 2 is formed by winding a metallic wire 21 into a helical coil structure around a longitudinal axis X of the catheter 1. In FIG. 1, the cylindrical main body 2 is formed by winding the metallic wire 21 into a left-handed helical coil structure toward the distal end of the catheter 1. However, the metallic wire 21 may be wound into a right-handed helical coil structure toward the distal end of the catheter 1. In FIG. 1, the metallic wire 21 is densely wound without forming spaces between adjacent windings of the metallic wire 21. However, as long as the form of the cylindrical main body 2 is maintained, the metallic wire 21 may be loosely wound so that spaces form between adjacent windings of the metallic wire 21, or a densely wound section and a loosely wound section may be combined.

Furthermore, the cylindrical main body 2 does not need to be formed by winding a metallic wire into a helical coil structure; the cylindrical coil body 2 may be formed by any method so long as the function as the cylindrical main body 2 is not deteriorated. For example, the cylindrical main body 2 may be formed by braiding metallic wires to form a braided coil structure.

The metallic wire 21 is a long linear body formed of metal. The metal forming the metallic wire 21 may be stainless steel, platinum, tungsten, or the like, for example. The cross section of the metallic wire 21 may be circular, oval, or rectangular, for example.

The metallic wire 21 forming the cylindrical main body 2 may be a single continuous piece or a plurality of pieces joined end-to-end. The metallic wire 21 may be formed entirely of the same material, or may be formed by combining metallic wire pieces of different materials. For example, the cylindrical main body 2 may be formed of only a metallic wire made of stainless steel, or may be formed of a combination of a radiotransparent metallic wire made of stainless steel and a radiopaque metallic wire made of platinum or tungsten.

The cylindrical main body 2 of the catheter 1 is formed by densely winding a single piece of metallic wire 21 having a circular cross section into a helical coil structure without forming any spaces between adjacent windings of the metallic wire 21.

The distal end tip 3 is formed at the distal end of the cylindrical main body 2 by melting and then solidifying a distal portion of the metallic wire 21 forming the cylindrical main body 2. Accordingly, the distal end tip 3 is not formed by joining a separate member to the cylindrical main body 2, but is instead formed from the metallic wire 21 itself (and thus both members are of the same material). In other words, the distal end tip 3 and the cylindrical main body 2 form a unitary (one-piece) structure. Therefore, the distal end tip 3 and the cylindrical main body 2 are integrally formed. This effectively prevents the distal end tip 3 from separating from the cylindrical main body 2 even under application of large torque forces.

The distal end tip 3 is formed at the distal end of the cylindrical main body 2 without closing the lumen 11. The distal end tip 3 is formed from the distal end of the catheter 1 and extends a length L toward the proximal end of the catheter 1 in the longitudinal direction. The length L may be 0.2 mm as a lower limit and 10 mm as an upper limit. When the length of the distal end tip 3 is in such a range, the distal end of the catheter 1 has excellent passing performance into an occluded portion, a stenosed portion, or the like, as well as excellent rigidity.

Furthermore, the flexibility at the distal end of the catheter 1 is not deteriorated. In a conventional catheter, the distal end tip is attached to the cylindrical main body as a separate member, and thus the length of the distal end tip must be longer to secure workability and connection strength, for example. By contrast, the distal end tip 3 of the catheter 1 is formed by melting the distal portion of the metallic wire 21. Thus, the distal end tip 3 does not need to be as long, and the flexibility at the distal end of the catheter 1 is not deteriorated.

Examples of methods of forming the distal end tip 3 include a method in which the metallic wire 21 is wound around a mandrel to form the cylindrical main body 2 around the mandrel; the distal end tip 3 is formed by heating the distal end of the cylindrical main body 2 wound around the mandrel by laser irradiation, for example, to melt the metallic wire 21 in a desired region; the melted metallic wire 21 at the distal end is cooled naturally or forcedly to solidify the melted metallic wire 21 and form the distal end tip 3; and the mandrel is finally removed. The shape or texture of the outer peripheral surface of the distal end tip 3 may be modified by grinding or polishing, for example.

When the heating is performed by laser irradiation, as described above, it is possible to easily target a specific area of the metallic wire 21 to be melted. Moreover, when the mandrel is used to form the distal end tip 3, the lumen 11 of the catheter 1 is not closed by melted metal of the metallic wire 21, and thus the distal end tip 3 is formed securely.

As the connector 4, a connector conventionally connected to proximal ends of various catheters is applied as needed. A guide wire, another catheter, or the like, which are used together with the catheter 1 in treatment, are introduced to the lumen 11 through an opening of the connector 4.

Each of the cylindrical main body 2 and the distal end tip 3 may be coated with resin on the outer surface, the inner surface, or both of the outer surface and the inner surface thereof. The coating may be applied to an entire surface, or applied to only a portion of the surface. When the coating is performed, the distal end tip 3 is formed before coating. In this way, cylindrical main body 2 and the distal end tip 3 may be integrally formed without the coating resin interfering with the process.

The resin forming the coating may be polytetrafluoroethylene, polyamide, or polyvinylpyrrolidone, for example.

In use, a guide wire preliminarily intended to be introduced to a lumen of a blood vessel or the like is inserted into the lumen 11 of the catheter 1, and the catheter 1 is introduced to the lumen of the blood vessel or the like along the guide wire. The catheter 1 is then passed through the lumen of the blood vessel or the like until reaching a target region such as an occluded portion or a stenosed portion. After reaching the target region, the catheter 1 is rotated or moved forward or backward while the distal end thereof (the distal end tip 3) is in contact with the occluded or stenosed portion, so that the distal end tip 3 enters into the occluded or stenosed portion. Once the distal end tip penetrates the occluded or stenosed portion, the catheter 1 is repeatedly rotated or moved forward or backward so that the cylindrical main body 2 of the catheter 1 passes through the occluded or stenosed portion. In this manner, the catheter 1 penetrates the target region and restores the functions of the blood vessel. If necessary, a device such as a balloon catheter or a stent, which is used together with the catheter 1, is introduced to the target region through the lumen 11 for treatment. Thereafter, the catheter 1 is withdrawn from the body.

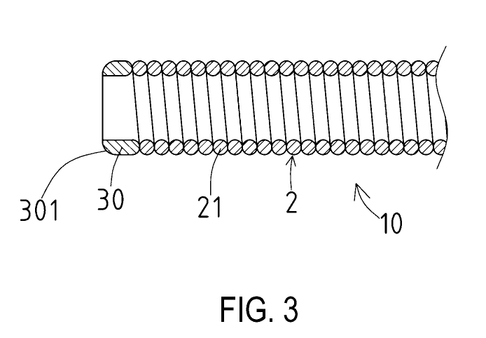

FIG. 3 is a cross-sectional view illustrating a distal end of a catheter (catheter 10) of the disclosed embodiments.

The catheter 10 has the same structure as the catheter 1 except that a distal end tip 30 of the catheter 10 has a different configuration than the distal end tip 3 of the catheter 1. Components that are the same are represented with the same symbols in the drawings.

The catheter 10 includes the hollow cylindrical main body 2 as in the catheter 1, and the distal end tip 30 is formed at the distal end of the cylindrical main body 2.

The distal end tip 30 is formed at the distal end of the cylindrical main body 2 by melting and then solidifying a distal portion of the metallic wire 21 forming the cylindrical main body 2, similarly to the distal end tip 3 of the catheter 1.

The distal end tip 30 has a tapered surface 301 formed by grinding or polishing, for example, an outer peripheral surface of a distal end of the distal end tip 30. With the tapered surface 301, an outer diameter of the distal end tip 30 is reduced toward the distal end, which further improves the passing performance of the distal end tip 30 into an occluded portion, a stenosed portion, or the like.

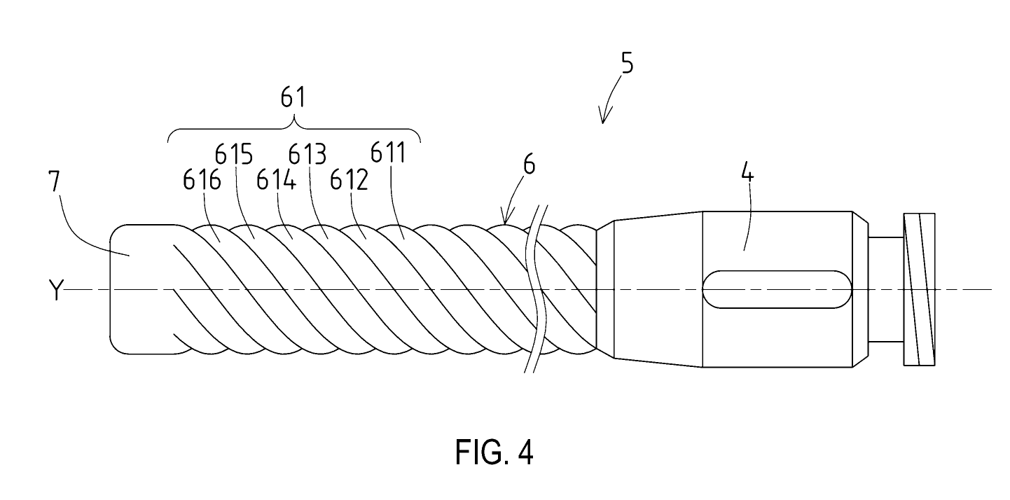

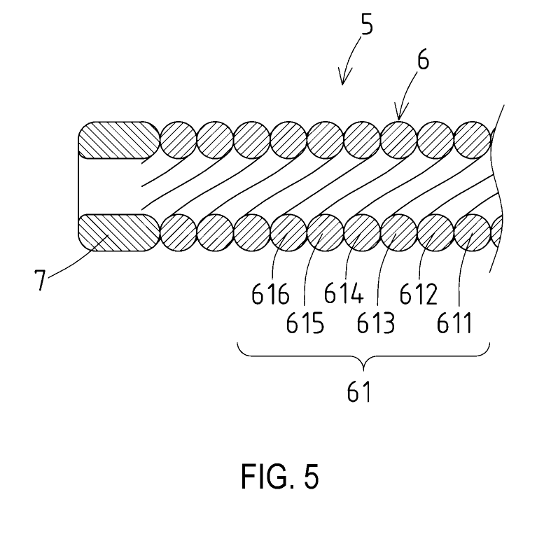

FIG. 4 and FIG. 5 are diagrams illustrating a catheter (catheter 5) of the disclosed embodiments. FIG. 4 is a side view of the catheter 5, and FIG. 5 is a cross-sectional view illustrating a distal end of the catheter 5.

The catheter 5 includes a hollow cylindrical main body 6, and a distal end tip 7 formed at a distal end of the cylindrical main body 6. A connector 4 is shown connected to a proximal end of the cylindrical main body 6. Similar to the connector 4 of the catheter 1, the connector 6 is not inserted into the body during operation.

The cylindrical main body 6 is the same as the cylindrical main body 2 of the catheter 1, except for the number of metallic wires forming the cylindrical main body.

The cylindrical main body 6 may be formed of stainless steel, and is formed by densely winding six metallic wires 61 (611, 612, 613, 614, 615, 616) having a circular cross section into a right-handed helical coil structure toward the distal end of the catheter 5 around a longitudinal axis Y of the catheter 5. Note that the metallic wires 61 may alternatively be wound into a left-handed helical coil structure toward the distal end. Additionally, the cylindrical main body 6 may include a loosely wound section.

In FIGS. 4 and 5, catheter 5 includes six metallic wires 61 (611, 612, 613, 614, 615, 616). However, the catheter 5 may include more or less than six metallic wires 61, as long as a plurality of metallic wires 61 is used. By using a plurality of the metallic wires 61, the flexibility of the obtained cylindrical main body 6 is further improved. As a result, there is an increased difference in flexibility between the cylindrical main body 6 and the distal end tip 7, and stress is easily concentrated at the junction between the cylindrical main body 6 and the distal end tip 7. In the conventional catheter having the metallic tip and the cylindrical main body as separate members, this would further increase the risk of the distal end tip separating from the cylindrical main body. In the catheter 5, however, the distal end tip 7 and the cylindrical main body 6 are integrally formed, thereby preventing the distal end tip 7 from separating from the cylindrical main body 6.

The cylindrical main body 6 is formed from at least 2 metallic wires 61. The cylindrical main body 6 is preferably formed from at least 6 metallic wires 61, and more preferably from at least 10 metallic wires 61. Additionally, the cylindrical main body 6 is preferably formed from no more than 30 metallic wires 61, and more preferably no more than 20 metallic wires 61. For example, the cylindrical main body 6 may be formed from 10 to 20 metallic wires 61. When the number of the metallic wires 61 is in such a range, the flexibility is optimally improved while maintaining passing performance and rigidity.

The distal end tip 7 is formed at the distal end of the cylindrical main body 6 by melting and then solidifying a distal portion of the metallic wires 61 forming the cylindrical main body 6, similarly to the method for forming the distal end tip 3 of the catheter 1.

Similar to the distal end tip 3 of the catheter 1, the distal end tip 7 is integrally formed with the cylindrical main body 6, thus effectively preventing the distal end tip 7 from separating from the cylindrical main body 6. In addition, the cylindrical main body 6 is formed of a plurality of the metallic wires 61, whereby the number of the ends of the metallic wires 61 that are positioned at the distal end of the cylindrical main body 6 is increased by the number of metallic wires 61 used. Therefore, the number of the metallic wires 61 joined to each other by melting during formation of the distal end tip 7 is increased, and the distal end tip 7 is formed easily and more securely.

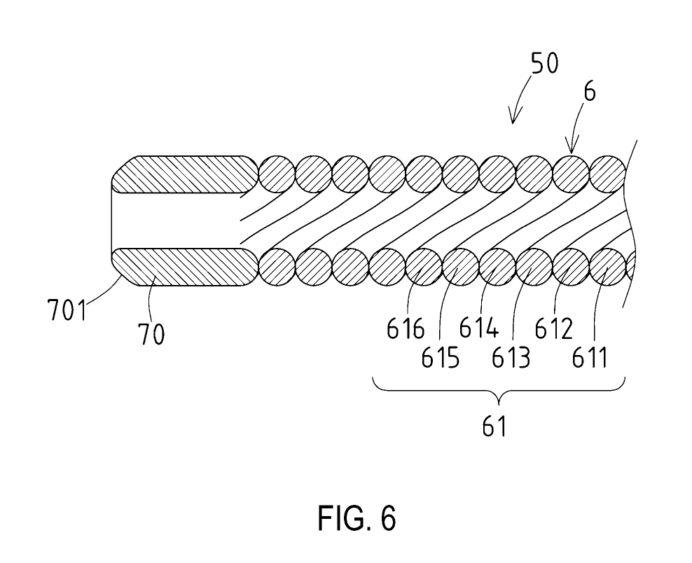

FIG. 6 is a cross-sectional view illustrating a distal end of a catheter (catheter 50) of the disclosed embodiments.

The catheter 50 has the same structure as the catheter 5 except that a distal end tip 70 of the catheter 50 has a different configuration than the distal end tip 7 of the catheter 5. Components that are the same are represented with the same symbols in the drawings.

The catheter 50 includes the hollow cylindrical main body 6 as in the catheter 5, and the distal end tip 70 is formed at the distal end of the cylindrical main body 6.

The distal end tip 70 is formed at the distal end of the cylindrical main body 6 by melting and then solidifying the distal portion of the metallic wires 61 forming the cylindrical main body 6, similarly to the distal end tip 7 of the catheter 5.

The distal end tip 70 has a tapered surface 701 formed by grinding or polishing, for example, an outer peripheral surface of a distal end of the distal end tip 70. With the tapered surface 701, an outer diameter of the distal end tip 70 is reduced toward the distal end, which further improves the passing performance of the distal end tip 70 into an occluded portion, a stenosed portion, or the like.

Moreover, the distal end tip 70 has a longer length than the distal end tip 7 of the catheter 5. The length of the distal end tip 70 may be appropriately adjusted in a range where the flexibility of a distal end of the catheter 50 is not deteriorated. When the distal end tip 70 has a longer length, the tapered surface 701 can be formed easily, whereby it is possible to securely improve the passing performance of the distal end tip 70 into an occluded portion, a stenosed portion, or the like.

What is claimed is:

1. A catheter comprising:
a hollow cylindrical main body comprising at least one metallic wire; and
a hollow distal end tip that is integrally formed with a distal end of the hollow cylindrical main body.

2. The catheter according to claim 1, wherein the hollow cylindrical main body is a helical coil structure formed by winding the at least one metallic wire around a longitudinal axis of the catheter.

3. The catheter according to claim 1, wherein the hollow distal end tip is formed by melting a distal portion of the at least one metallic wire of the hollow cylindrical main body.

4. The catheter according to claim 1, wherein the hollow cylindrical main body comprises a plurality of the metallic wires.

5. The catheter according to claim 4, wherein the hollow cylindrical main body is formed by winding the plurality of the metallic wires around a longitudinal axis of the catheter.

6. The catheter according to claim 5, wherein the hollow distal end tip is formed by melting a distal portion of the plurality of the metallic wires of the hollow cylindrical main body.

7. The catheter according to claim 1, wherein the hollow cylindrical main body comprises a densely wound section that does not include any spaces between adjacent windings of the at least one metallic wire.

8. The catheter according to claim 1, wherein the hollow cylindrical main body comprises a loosely wound section that includes spaces between adjacent windings of the at least one metallic wire.

9. The catheter according to claim 1, wherein the hollow distal end tip has a tapered outer peripheral surface.

10. The catheter according to claim 1, wherein the hollow distal end tip and the hollow cylindrical main body are of the same material.

11. The catheter according to claim 1, wherein the hollow distal end tip and the hollow cylindrical main body form a unitary structure.

12. The catheter according to claim 1, wherein the hollow distal end tip is comprised of multiple windings of the at least one metallic wire melted together.
